(19) United States
(12) Patent Application Publication
(10) Pub. No.: US 2026/0043001 A1
(43) Pub. Date: Feb. 12, 2026

(54) DECELLULARIZED AMNIOTIC MEMBRANE MATRIX HYDROGEL AND METHOD OF PREPARATION THEREOF

(71) Applicant: Tongji Hospital, Tongji Medical College of HUST, Wuhan City (CN)

(72) Inventors: Guigang LI, Wuhan City (CN); Yongyao TAN, Wuhan City (CN); Wei WANG, Wuhan City (CN); Lingjuan XU, Wuhan City (CN); Yunming WANG, Wuhan City (CN)

(21) Appl. No.: 18/919,690

(22) Filed: Oct. 18, 2024

(30) Foreign Application Priority Data
Aug. 12, 2024 (CN) 202411105273.2

(51) Int. Cl. C12N 5/073; A01N 1/02

(52) U.S. Cl. CPC C12N 5/0605; A01N 1/128; C12N 2533/92

(57) ABSTRACT

Provided are a decellularized amniotic membrane matrix hydrogel and its preparation method, which includes the following steps: pre-treatment of an amniotic membrane tissue, washing, decellularization, grinding, freeze-drying, digestion, and acid-base neutralization processes. The decellularized amniotic membrane matrix hydrogel provided in this disclosure retains components such as collagen, fibronectin, and glycoproteins from the amniotic membrane while removing viable cells, thereby reducing its immunogenicity at the source. The decellularized amniotic membrane matrix hydrogel may mimic the microenvironment in which cells exist in vivo, providing the necessary support and signals for cell adhesion and growth. It can be used to construct tissue engineering scaffolds, supporting directed cell growth and tissue regeneration. Furthermore, the decellularized amniotic membrane matrix hydrogel may also serve as a drug carrier for in vivo drug release and therapy, demonstrating significant potential for application in medical research and clinical settings.

### CROSS-REFERENCE TO RELATED APPLICATION

This patent application claims the benefit and priority of Chinese Patent Application No. 2024111052732 filed with the China National Intellectual Property Administration on Aug. 12, 2024, the disclosure of which is incorporated by reference herein in its entirety as part of the present application.

### TECHNICAL FIELD

The present disclosure relates to the field of hydrogel technology, specifically to a decellularized amniotic membrane matrix hydrogel and a method of preparation thereof.

### BACKGROUND

Recently, fully synthetic hydrogels have been widely explored for three-dimensional cell culture, including organoids. Their advantages lie in the stability of synthetic materials and the uniformity of their structure. However, fully synthetic materials often lack the growth factors necessary for cell proliferation and exhibit poor biocompatibility. Consequently, the cell culture performance of fully synthetic hydrogels is often significantly inferior to that of biologically derived matrix hydrogels.

Matrix hydrogels are composite materials composed of soluble extracellular matrix components, which include collagen, laminin, and fibronectin, among others. Matrix hydrogels play an important role in biological experiments and have a wide range of applications. Firstly, they can be used to treat cell culture plates, facilitating cell attachment and growth. Secondly, they can be laid in Transwell chambers to study the invasive capabilities of tumor cells. Additionally, they can be used for three-dimensional culture of cell lines or patient-derived cells, thereby forming organoid structures that provide models for anticancer drug screening. Currently, Engelbreth-Holm-Swarm (EHS) matrix hydrogels are one of the main types; these hydrogels are primarily extracted from EHS cells. However, EHS cells themselves cannot be cultured in vitro, which limits their scalability for production and poses significant challenges for the mass production of matrix hydrogels. Furthermore, the preparation of EHS matrix hydrogels requires the inoculation of osteosarcoma in mice, and this tumor-derived characteristic severely restricts the clinical application prospects of related cell therapies.

The amniotic membrane is a natural membrane surrounding the fetus and is an important tissue formed during embryonic development. It is rich in collagen, elastic fibers, and hyaluronic acid, possessing good biocompatibility and biological activity. It can be used in tissue repair for corneal and skin transplants, making it an ideal source of extracellular matrix.

### SUMMARY

In light of this, the present disclosure provides a method for preparing a decellularized amniotic membrane matrix hydrogel, including the following steps:
(1) pre-treating and decellularizing an amniotic membrane: freezing a fresh amniotic membrane in liquid nitrogen, taking out a frozen amniotic membrane for thawing, repeating a freezing-thawing process for 3-5 cycles to obtain a decellularized amniotic membrane;
(2) freezing the decellularized amniotic membrane from step (1) again in liquid nitrogen, followed by grinding and sieving to obtain decellularized amniotic membrane powder;
(3) freeze-drying the decellularized amniotic membrane powder obtained in step (2) to yield freeze-dried decellularized amniotic membrane powder; and
(4) mixing the freeze-dried decellularized amniotic membrane powder from step (3) with pepsin, then adding a 0.01 M hydrochloric acid solution for digestion; after the digestion, lowering an environmental temperature to 0-4° C., adjusting a pH of a system to neutral to obtain a mixed solution; finally, adding a phosphate-bufferred saline (PBS) solution to the mixed solution and allowing a resulting mixture to stand to obtain the decellularized amniotic membrane matrix hydrogel.

In some embodiments, in step (1), the freezing is conducted for 10-15 minutes, and the melting is performed at 37-40° C.

In the present disclosure, freezing is performed to form ice particles inside the cells, which are then thawed at room temperature (or 40° C.). The freezing increases the salt concentration of the remaining cytosol, causing cell swelling and rupture, thereby reducing the amount of reagents needed.

In some embodiments, in step (1), pre-treating the amniotic membrane specifically includes: collecting a fresh amniotic membrane tissue from postpartum women aged 25 to 35 years who have been tested negative for infectious diseases through serological screening, cleaning the fresh amniotic membrane tissue with sterile physiological saline, removing chorions, and cutting the fresh amniotic membrane tissue into small pieces of 2×2 cm; conducting rinsing in sterile physiological saline at 0-4° C. for 30-35 minutes at a rotation speed of 150-200 rpm, followed by rinsing with deionized water at 0-4° C. for another 30-35 minutes at the rotation speed of 150-200 rpm.

In step (2), the sieving is performed using a 200-mesh sieve.

In step (3), the freeze-drying is conducted at −70 to −80° C., with a freeze-drying duration of 36-48 hours.

In step (4), a mass ratio of the freeze-dried decellularized amniotic membrane powder to the pepsin is 10-14:3.

In step (4), an amount of the PBS solution used is 9-10 times that of the mixed solution.

Furthermore, the present disclosure also provides a decellularized amniotic membrane matrix hydrogel obtained by the method, as well as its use in cell culture, tissue engineering, and drug delivery systems.

Compared to the prior art, the embodiments of the present disclosure offers the following beneficial effects.

In this disclosure, decellularization technology and hydrogel preparation techniques are introduced to produce a decellularized amniotic membrane matrix hydrogel. The resulting decellularized amniotic membrane matrix hydrogel retains components such as collagen, fibronectin, and glycoproteins from the amniotic membrane while removing viable cells, thereby reducing its immunogenicity at the source. In cell culture, this hydrogel can mimic the microenvironment in which cells exist in vivo, providing the necessary support and signals for cell adhesion and growth, which aids in the in vitro culture and expansion of cells. In tissue engineering, the decellularized amniotic membrane matrix hydrogel can be used to construct tissue engineering scaffolds, supporting directed cell growth and tissue regeneration. Moreover, this decellularized amniotic membrane matrix hydrogel can also serve as a drug carrier for in vivo drug release and therapy. Due to its excellent biocompatibility and biological activity, the decellularized amniotic membrane matrix hydrogel holds significant potential in medical research and clinical applications.

### BRIEF DESCRIPTION OF THE DRAWINGS

FIG. 1 is a graph showing the comparison of effects between the decellularized amniotic membrane (dAM) prepared in Example 1 of the present disclosure and the fresh amniotic membrane (fAM) from Comparative Example 1.

FIG. 2 is a graph showing the comparison of effects between the decellularized amniotic membrane matrix hydrogel prepared in Examples 1 and 2 of the present disclosure and the Matrigel hydrogel from Comparative Example 2.

FIG. 3 is an electron microscopy image showing the comparison between the decellularized amniotic membrane matrix hydrogel prepared in Example 1 of the present disclosure and the Matrigel hydrogel from Comparative Example 2.

### DETAILED DESCRIPTION OF THE EMBODIMENTS

To make the objectives, technical solutions, and advantages of the present disclosure clearer, the following provides a detailed and complete description of the technical solutions of the disclosure. It is evident that the described examples are only a part of the embodiments of the present disclosure and not all possible embodiments. Any other embodiments derived by those skilled in the art based on the examples of the present disclosure, without making any inventive effort, fall within the protection scope of the present disclosure.

In the following examples, the experimental methods or testing methods, unless otherwise specified, are conventional methods; the raw materials and additives, unless otherwise specified, are obtained from conventional commercial sources or prepared by conventional methods.

### I. Measurement of DNA Content

1. A sample of 20 mg is taken and frozen in liquid nitrogen, then ground or crushed.
2. After the sample is processed, 5 μL of proteinase K is added to each 1 mL of sample lysis buffer and mixed thoroughly.
3. For the prepared sample, 500 μL of the proteinase K-treated sample lysis buffer is added to the 20 mg of tissue, and the mixture is inverted and mixed several times to ensure complete lysis of the tissue or cells.
4. The mixture is incubated in a 50° C. water bath overnight for digestion.
5. After digestion, 500 μL of Tris-balanced phenol extraction solution is added to the sample.
6. The phenol and interphase are aspirated (a small amount of the aqueous phase near the interphase can be removed), and the remaining aqueous phase is extracted with an equal volume of Tris-balanced phenol once more.
7. Again, the phenol and interphase are aspirated (a small amount of the aqueous phase near the interphase can be removed), and the remaining aqueous phase is extracted with an equal volume of chloroform.
8. About 300 μL of the supernatant is aspirated, and 60 μL of 10 M ammonium acetate and 600 μL of absolute ethanol are added, followed by several inversions to mix; at this point, DNA precipitation can be observed.
9. The mixture is centrifuged at 20,000 rpm for 1 minute, and the supernatant is discarded. The DNA pellet is washed twice with 70% ethanol.
10. Residual ethanol is carefully removed, and once no visible liquid remains, 50-100 μL of ultrapure water is immediately added to dissolve the DNA.
11. The DNA concentration is measured using a micro-volume spectrophotometer at a wavelength of 280 nm.

### II. Measurement of Collagen Content

Hydroxyproline (HYP) is one of the main components of collagen in the body, primarily distributed in tissues such as skin, tendons, cartilage, and blood vessels. Therefore, the HYP content serves as an important indicator of collagen tissue metabolism and the degree of fibrosis. The sample undergoes acid hydrolysis to produce free HYP, which is then oxidized by chloramine T. The oxidation products react with para-dimethylaminobenzaldehyde to form a red compound that exhibits a characteristic absorption peak at 560 nm. By measuring the absorbance of the sample hydrolysate at 560 nm, the HYP content can be calculated.

### Extraction:

Approximately 0.2 g of the sample is weighed and placed in a glass tube. The tissue is cut into small pieces to facilitate digestion, and the cap is left slightly loose to prevent sealing tightly. Two milliliters of extraction solution (6 mol/L hydrochloric acid solution) is added, and the mixture is boiled or placed in a 110° C. oven for digestion for 2 to 6 hours until no large visible clumps remain (the cap is covered with sealing film to prevent explosion). After cooling, the pH is adjusted to the range of 6-8 using NaOH solution (approximately 1 mL of 10 mol/L NaOH is needed, ensuring it is not too acidic or too alkaline), and the volume is made up to 4 mL with distilled water. The mixture is then centrifuged at 16,000 rpm for 20 minutes at 25° C., and the supernatant is collected for measurement.

### Measurement:

1. The spectrophotometer is preheated for more than 30 minutes, and the wavelength is set to 560 nm, with distilled water used to calibrate the instrument.
2. A 0.5 mg/mL standard solution of hydroxyproline is prepared and diluted with distilled water to create standard solutions with concentrations of 15, 7.5, 3.75, 1.875, 0.938, 0.469, 0.234, and 0.117 μg/mL.
3. The samples are divided into three groups: blank tube, measurement tube, and standard tube. In the blank tube, 200 μL of chloramine T is added; in the measurement tube, 200 μL of the sample and 200 μL of chloramine T are added; and in the standard tube, 200 μL of the standard solution and 200 μL of chloramine T are added. The mixtures are mixed thoroughly and allowed to stand at 25° C. for 20 minutes. Afterward, 200 μL of para-dimethylaminobenzaldehyde and 600 μL of distilled water are added to the blank tube, 200 μL of para-dimethylaminobenzaldehyde and 400 μL of distilled water to the measurement tube, and 200 μL of para-dimethylaminobenzaldehyde and 400 μL of distilled water to the standard tube.

The contents are mixed and incubated in a 60° C. water bath for 20 minutes. After incubation, the tubes are allowed to stand at room temperature for 15 minutes. The absorbance values of the blank tube, measurement tube, and standard tube are measured at 560 nm using a 1 mL glass cuvette, recorded as A_{blank}, Aₘₑₐₛᵤᵣₑₘₑₙₜ, and A_{standard}, respectively. The changes in absorbance are calculated as ΔAₘₑₐₛᵤᵣₑₘₑₙₜ=Aₘₑₐₛᵤᵣₑₘₑₙₜ−A_{blank} and ΔA_{standard}=A_{standard}−A_{blank}.
4. A standard curve is plotted with the concentration of the standard solution on the x-axis and ΔA_{standard} on the y-axis, yielding the equation y=kx+b. The value of ΔAₘₑₐₛᵤᵣₑₘₑₙₜ is substituted into the equation to determine x (μg/mL).
5. The hydroxyproline content in the tissue is calculated using the following formula: Tissue Hydroxyproline Content (μg/g)=x×Vₛₐₘₚₗₑ÷(W×Vₛₐₘₚₗₑ÷V_{tissue extract})=4x÷W, where:
Vₛₐₘₚₗₑ: volume of the added sample, 0.2 mL; V_{tissue extract}: volume of the tissue extract, 4 mL; and W: mass of the sample (g).

### III. Measurement of Glycosaminoglycans (GAG)

Glycosaminoglycans (GAGs), also known as mucopolysaccharides, are the most abundant heteropolysaccharides in the body. GAGs are long-chain polysaccharides that are unbranched and negatively charged, composed of repeating disaccharide units that include hexoses or hexuronic acids linked to hexosamines. GAGs covalently bind to core proteins to form proteoglycans (PGs), which are major components of cell membranes and the extracellular matrix (ECM). During their synthesis, GAGs undergo sulfation at O or N positions, which facilitates various functions, including the regulation of enzymes, cell adhesion, growth, migration, differentiation, and responses to tissue injury. Due to the highly negative charge of GAGs, the metachromatic cationic dye, 1,9-dimethylmethylene blue (DMMB), is used under acidic conditions to specifically bind to GAGs, forming insoluble purple or pink dye-GAG complexes. In a dissociation solution, the dye is released and can be quantified using a microplate reader at a wavelength of 530 nm to analyze the total GAG content.

### I. Preparation of DMMB Reagent

1. Sixteen micrograms of DMMB is added to a beaker containing 5 mL of ethanol and stirred, while the mixture is wrapped with aluminum foil to protect it from light.
2. Three and four hundred grams of glycine, 2.37 g of NaCl, and 95 mL of 0.1 N HCl are added.
3. Eight hundred milliliters of distilled water is added, and the pH is adjusted to 3.0 using 0.1 N HCl.
4. The volume is made up to 1000 mL with distilled water.
5. The mixture is stirred at room temperature using a magnetic stirrer for 2 to 16 hours, while being protected from light.
6. Impurities are removed using filter paper with a pore size of 20 μm to 25 μm.

### II. Measurement of Samples

1. Ten milligrams of 6-sulfated chondroitin (purity>95%) is added to 20 mL of extraction liquid to prepare a 0.5 mg/mL solution, which is stirred for several minutes until full dissolution.
2. A series of standard chondroitin sulfate solutions are prepared using the extraction liquid at the following concentrations: 0 μg/mL, 3.125 μg/mL, 6.25 μg/mL, 12.5 μg/mL, 25 μg/mL, 50 μg/mL, and 100 μg/mL.
3. The microplate reader is turned on.
4. Two hundred milliliters of the extracted sample or the standard chondroitin sulfate solution is added to 0.2 mL of DMMB solution, mixed thoroughly, and the absorbance is measured at 530 nm.

### III. Normalization of GAG Content

The absorbance is converted into GAG concentration (μg/mL), which is then multiplied by the volume of the digestion liquid added to each sample and the dilution factor to obtain the sGAG content (μg) in the sample.

The sGAG content (μg) is divided by the wet weight of the parallel test samples to normalize it to the unit mass of sGAG content (μg/mg). The formula used is: sGAG (μg/mg)=A×B×C/D, where
A is the GAG concentration (μg/mL);
B is the dilution factor;
C is the volume of the test sample (volume of extraction liquid+volume of parallel sample), mL; and
D is the wet weight of the parallel test sample, mg.
   IV. Method for culturing Limbal Niche Cells (LNCs) and Proliferation Measurement

### 1. Specimen Acquisition

Specimens containing intact corneal and limbal tissues are selected and cut radially into equal-sized limbal pieces.

### 2. Culturing

The limbal pieces are digested in 1 mL of collagenase A solution at 37° C. in a 5% CO₂ environment for 8.0 to 10.0 hours. After digestion, 1 mL of trypsin solution is added for an additional 10 minutes. The digested cells are then seeded onto a six-well plate coated with gel (decellularized amniotic membrane hydrogel/Matrigel mixed with modified embryonic SC medium (MESCM) at a 5% ratio on ice before being placed in an incubator to complete the gel coating). Primary limbal niche cells are obtained and subsequently passaged to obtain the fourth generation of limbal niche cells. The trypsin solution contains 0.25% trypsin and ethylene diamine tetraacetic acid (EDTA) in PBS (pH 7.2-7.4), with a digestion time of 10 to 20 minutes; the mass fraction of trypsin is 0.25%, and the mass fraction of EDTA is 0.02%.

The modified embryonic SC medium (MESCM) is prepared according to the following table.

| Reagent | Sources | Amount |
|---|---|---|
| CTS KnockOut Dulbecco's Modified | Gibco | DMEM, the basal |
| Eagle Medium (DMEM) | | medium, mixed with |
| | | F-12 culture medium |
| | | in a 1:1 ratio, with |
| | | the DMEM basal |
| | | medium weighing |
| | | 40-50 g |
| CTS ™ KnockOut ™ SR XenoFree | Gibco | 10% |
| Specific Knockout Serum Replacement | | |
| PeproGMP ® Recombinant Human | PEPRO | 4 ng/ml |
| Fibroblast Growth Factor (FGF)-basic | TECH | |
| PeproGMP ® Recombinant Human | PEPRO | 10 ng/ml |
| Leukemia Inhibitory Factor (LIF) | TECH | |

### 3. Cell Seeding

A final concentration of 3000 cells/100 μL is seeded into each well with the fourth-generation limbal niche cells (LNCs), with five replicates set for each concentration. Additionally, blank wells containing 100 μL of MESCM are included. Cell density is observed every 12 hours. Three 96-well plates are simultaneously seeded, and PBS is added around the experimental area to prevent evaporation of the culture medium in the incubator.

### 4. Detection

On days 1, 3, and 5, a mixed solution (10 μL of CCK-8 solution+90 μL of MESCM) is added in each well to replace previous solutions. Air bubbles are pierced using a sterilized needle that has been burned. The mixture is incubated in the dark at 37° C. for 3 hours, and the absorbance at 450 nm is measured using a microplate reader.

### Example 1

A method for preparing decellularized amniotic membrane matrix hydrogel, including the following steps:
(1) Amniotic membrane tissue pre-treatment: fresh amniotic membrane tissues were collected from postpartum women aged 25 to 35 years, who had tested negative for infectious diseases through serological screening. The tissues were cleaned with sterile physiological saline, the chorion was removed, and the membranes were cut into small pieces measuring 2 cm×2 cm.
(2) Washing: the fresh amniotic membranes were placed in physiological saline at 4° C. and rinsed for 30 minutes at a rotational speed of 150 rpm. They were then rinsed again in deionized water at 4° C. for another 30 minutes at a rotational speed of 150 rpm.
(3) Decellularization: the washed amniotic membranes were frozen in liquid nitrogen for 10 minutes, followed by thawing in a 37° C. water bath for 10 minutes. This process was repeated three times, and the membranes were subsequently washed with sterile physiological saline to obtain decellularized amniotic membranes.
(4) Grinding: the decellularized amniotic membranes were again frozen in liquid nitrogen for 10 minutes, then ground in a mortar. The ground membranes were passed through a 200-mesh sieve to obtain decellularized amniotic membrane powder.
(5) Freeze-drying: the decellularized amniotic membrane powder was placed in a freeze dryer and freeze-dried at −80° C. for 48 hours to yield freeze-dried decellularized amniotic membrane powder.
(6) Digestion: in a 25° C. environment, the freeze-dried decellularized amniotic membrane powder (14 mg) was mixed with pepsin (1:3000, 3 mg) in 0.01 M HCl, and the volume was adjusted to 1 mL. The mixture was continuously agitated for 96 hours to achieve digestion, resulting in a digested decellularized amniotic membrane solution.
(7) pH neutralization: in a 4° C. environment, 0.1 M NaOH was added to the decellularized amniotic membrane solution until the pH reached 7. Subsequently, a 10-fold concentration of PBS was added, and the mixture was allowed to stand to obtain the decellularized amniotic membrane matrix hydrogel.

### Example 2

The difference from Example 1 lied in the amount of freeze-dried decellularized amniotic membrane powder added, which was 10 mg, while all other steps remained consistent with Example 1.

### Comparative Example 1

Fresh amniotic membrane that had not been treated was used.

### Comparative Example 2

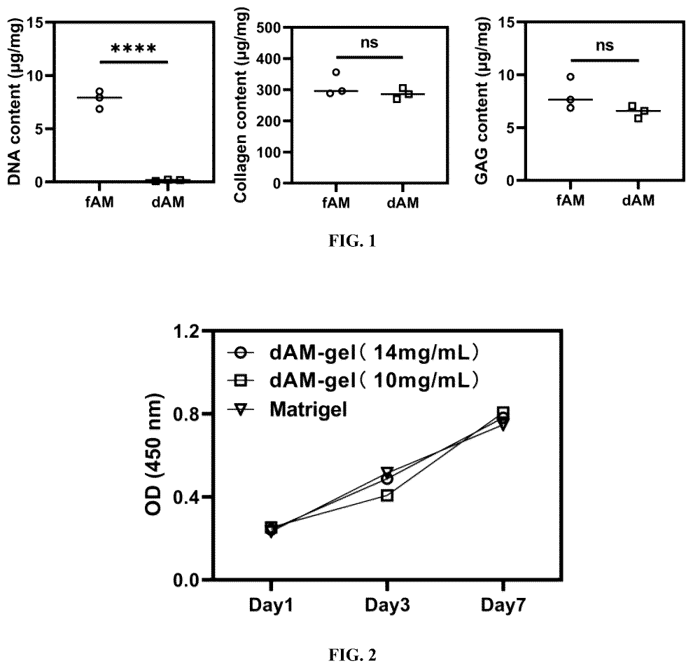

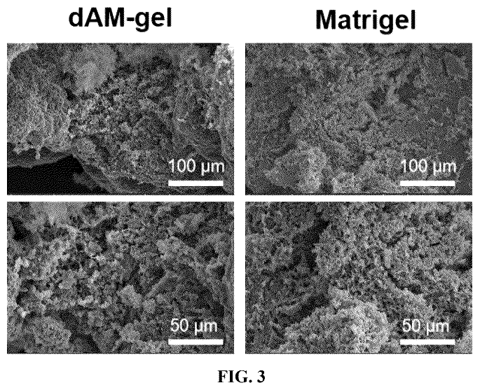

Matrigel hydrogel was used.
(1) FIG. 1 presents a comparison of the effects of decellularized amniotic membrane (dAM) prepared in Example 1 with fresh amniotic membrane (fAM) from Comparative Example 1. By measuring the DNA content, collagen content, and glycosaminoglycan (GAG) content before and after treatment of the amniotic membrane, it was observed that the DNA content in the decellularized amniotic membrane was significantly reduced compared to that of the fresh amniotic membrane (7.73±0.86 μg/mg vs 0.16±0.07 μg/mg). Additionally, the majority of collagen (313.00±37.40 μg/mg vs 287.10±17.88 μg/mg) and GAG (dry weight 8.11=1.52 μg/mg vs 6.50±0.59 μg/mg) was retained in the decellularized amniotic membrane. Statistical significance was indicated (****P<0.001), demonstrating that the treatment method provided by the present disclosure achieves decellularization while preserving the majority of effective substances.
(2) FIG. 2 illustrates a comparison of the decellularized amniotic membrane matrix hydrogel prepared in Examples 1 and 2 with Matrigel hydrogel from Comparative Example 2. The biocompatibility of the decellularized amniotic membrane matrix hydrogel was assessed by culturing limbal niche cells (LNCs) on a six-well plastic plate coated with the hydrogel. It was found that the biocompatibility of the decellularized amniotic membrane matrix hydrogel provided by the present disclosure was similar to that of the Matrigel hydrogel. Moreover, no statistically significant differences in optical density (OD) values among the three hydrogels were observed within 7 days, indicating that the ability to promote LNC proliferation was comparable between the decellularized amniotic membrane matrix hydrogel and the Matrigel hydrogel. Thus, it can be concluded that the decellularized amniotic membrane matrix hydrogel provided by the present disclosure exhibits good biocompatibility and can promote the proliferation of limbal niche cells.
(3) FIG. 3 shows a comparison of the scanning electron microscopy images of the decellularized amniotic membrane matrix hydrogel prepared in Example 1 and the Matrigel hydrogel from Comparative Example 2. The scanning electron microscopy images reveal that the dried decellularized amniotic membrane matrix hydrogel and the Matrigel hydrogel possess a similar highly porous network structure, having interconnected pore networks that facilitate cell migration and proliferation. This structure also aids in the diffusion of nutrients and oxygen, as well as the removal of waste products.

The above descriptions represent preferred embodiments of the present disclosure. It should be noted that various modifications and refinements may be made by those skilled in the art without departing from the principles of the present disclosure. These modifications and refinements should also be considered within the scope of protection of the present disclosure.

What is claimed is:

1. A method for preparing a decellularized amniotic membrane matrix hydrogel, comprising steps of:
1) pre-treating and decellularizing an amniotic membrane: freezing a fresh amniotic membrane in liquid nitrogen, taking out a frozen amniotic membrane for thawing, repeating a freezing-thawing process for 3-5 cycles to obtain a decellularized amniotic membrane;
2) freezing the decellularized amniotic membrane from step 1) again in liquid nitrogen, followed by grinding and sieving to obtain decellularized amniotic membrane powder;
3) freeze-drying the decellularized amniotic membrane powder obtained in step 2) to yield freeze-dried decellularized amniotic membrane powder; and
4) mixing the freeze-dried decellularized amniotic membrane powder from step 3) with pepsin, then adding a 0.01 M hydrochloric acid solution for digestion; after the digestion, lowering an environmental temperature to 0-4° C., adjusting a pH of a system to neutral to obtain a mixed solution; finally, adding a phosphate-bufferred saline (PBS) solution to the mixed solution and allowing a resulting mixture to stand to obtain the decellularized amniotic membrane matrix hydrogel.

2. The method of claim 1, wherein the freezing time in step 1) is 10 minutes.

3. The method of claim 1, wherein the melting temperature in step 1) is 37° C.

4. The method of claim 1, wherein pre-treatment of the amniotic membrane tissue in step 1) specifically comprises: from postpartum women aged 25 to 35 years who have been tested negative for infectious diseases through serological screening, cleaning the fresh amniotic membrane tissue with sterile physiological saline, removing chorions, and cutting the fresh amniotic membrane tissue into small pieces of 2×2 cm; conducting rinsing in sterile physiological saline at 0-4° C. for 30-35 minutes at a rotation speed of 150-200 rpm, followed by rinsing with deionized water at 0-4° C. for another 30-35 minutes at the rotation speed of 150-200 rpm.

5. The method of claim 1, wherein the sieving in step 2) is performed using a 200-mesh sieve.

6. The method of claim 1, wherein the freeze-drying in step 3) is conducted at −70 to −80° C., with a freeze-drying duration of 36-48 hours.

7. The method of claim 1, wherein a mass ratio of the freeze-dried decellularized amniotic membrane powder to pepsin in step 4) is 10-14:3.

8. The method of claim 1, wherein an amount of PBS buffer solution used in step 4) is 10 times that of the mixed solution.

9. A decellularized amniotic membrane matrix hydrogel obtained by the method of claim 1.

10. The decellularized amniotic membrane matrix hydrogel of claim 9, wherein the freezing time in step 1) is 10 minutes.

11. The decellularized amniotic membrane matrix hydrogel of claim 9, wherein the melting temperature in step 1) is 37° C.

12. The decellularized amniotic membrane matrix hydrogel of claim 9, wherein pre-treatment of the amniotic membrane tissue in step 1) specifically comprises: from postpartum women aged 25 to 35 years who have been tested negative for infectious diseases through serological screening, cleaning the fresh amniotic membrane tissue with sterile physiological saline, removing chorions, and cutting the fresh amniotic membrane tissue into small pieces of 2×2 cm; conducting rinsing in sterile physiological saline at 0-4° C. for 30-35 minutes at a rotation speed of 150-200 rpm, followed by rinsing with deionized water at 0-4° C. for another 30-35 minutes at the rotation speed of 150-200 rpm.

13. The decellularized amniotic membrane matrix hydrogel of claim 9, wherein the sieving in step (2) is performed using a 200-mesh sieve.

14. The decellularized amniotic membrane matrix hydrogel of claim 9, wherein the freeze-drying in step 3) is conducted at −70 to −80° C., with a freeze-drying duration of 36-48 hours.

15. The decellularized amniotic membrane matrix hydrogel of claim 9, wherein a mass ratio of the freeze-dried decellularized amniotic membrane powder to pepsin in step 4) is 10-14:3.

16. The decellularized amniotic membrane matrix hydrogel of claim 9, wherein an amount of PBS buffer solution used in step 4) is 10 times that of the mixed solution.
